(19) United States
(12) Patent Application Publication
(10) Pub. No.: US 2018/0360352 A1
(43) Pub. Date: Dec. 20, 2018

(54) OPTOSENSOR

(71) Applicant: NEW JAPAN RADIO CO., LTD., Chuo-ku, Tokyo (JP)

(72) Inventor: Fumiaki OHNO, Fujimino-shi, Saitama (JP)

(21) Appl. No.: 15/780,028

(22) PCT Filed: Nov. 30, 2015
(86) PCT No.: PCT/JP2015/083654

§ 371 Date: May 30, 2018

(51) Int. Cl. A61B 5/1455; A61B 5/026; A61B 5/0245; G01N 21/35

(52) U.S. Cl. CPC A61B 5/1455; A61B 5/0261; A61B 2562/16; G01N 21/35; A61B 2562/0233; A61B 5/0245

(57) ABSTRACT

An optosensor includes an optoreflector (1) having a light emitting element (12) and a light receiving element (13) which are mounted on a substrate (11), a light transmitting resin (15) sealing the light emitting element (12) and the light receiving element (13), and a light blocking resin (2), and optionally further includes a light transmitting cover (20) that is disposed to face a light emitting/receiving portion of the optoreflector (1). In the optosensor, a light blocking wall (2a) and an eave (2b) are formed between the light emitting element (12) and the light receiving element (13) with the light blocking resin (2), the cave (2b) being linked to the light blocking wall (2a) to narrow an upper light emitting surface of the light emitting element (12).

### TECHNICAL FIELD

The present invention relates to an optosensor that measures a pulse wave or a heart rate by detecting biological information.

### BACKGROUND ART

In the healthcare industry, an optosensor is known which monitors biological information such as a pulse wave, a heart rate, or a blood oxygenation level by monitoring a change in hemoglobin concentration in blood pulsing through a blood vessel using a reflective optoreflector as a sensor head. Recently, instead of a stationary optosensor, a mobile or wearable optosensor has been developed. In particular, a wearable optosensor built into a bracelet, smartwatch, or an in-ear earphone has attracted much attention.

The mobile or wearable optosensor is frequently carried outdoors and thus is required to have a drip-proof or water-proof structure. In an example of the drip-proof or water-proof structure, an optoreflector is sealed into an air-tight housing, and biological information is monitored through a light transmitting protective member (cover) disposed on the optoreflector.

FIGS. 6(a) and 6(b) illustrate an optoreflector 10 of the related art used in a mobile or wearable optosensor. FIG. 6(a) is a plan view illustrating the optoreflector 10 when seen from a light emitting/receiving surface, and FIG. 6(b) is a cross-sectional view illustrating the optoreflector 10. The optoreflector 10 illustrated in FIGS. 6(a) and (b) includes a substrate 11, a light emitting element 12, a light receiving element 13, a light blocking resin 14, and a light transmitting resin 15. The substrate 11 is a printed circuit board that is formed of a copper clad laminate in which a rectangular base formed of glass epoxy or the like is coated with copper foil. On a back surface of the substrate 11, an external electrode 11a is formed. On a front surface of the substrate 11, a die pad 11b and a bonding pad 11c for the light emitting element 12, a die pad 11d and a bonding pad 11e for the light receiving element 13 are formed. The die pads 11b and 11d and the bonding pads 11c and 11e are electrically connected to the external electrode 11a through via holes (not illustrated).

As illustrated in FIGS. 6(a) and 6(b), two light emitting elements 12 are provided and mounted on opposite end portions of the substrate 11 in a longitudinal direction, respectively. The light receiving element 13 is mounted between the two light emitting elements 12. The light blocking resin 14 is formed around the substrate 11 and has a larger thickness than the light emitting elements 12 and the light receiving element 13. In addition, the light blocking resin 14 forms light blocking walls 14a that block light emitted from the two light emitting elements 12 from being directly incident on the light receiving element 13. That is, the light blocking walls 14a formed of a part of the light blocking resin 14 are formed between one light emitting element 12 and the light receiving element 13 and between the other light emitting element 12 and the light receiving element 13, respectively. The light blocking wall 14a between one light emitting element 12 and the light receiving element 13 prevents light from being directly incident on a gap between the light emitting element 12 and the light receiving element 13. In addition, the light blocking wall 14a between the other light emitting element 12 and the light receiving element 13 prevents light from being directly incident on a gap between the light emitting element 12 and the light receiving element 13.

In one light emitting element 12 and a periphery thereof, the light receiving element 13 and a periphery thereof, and the other light emitting element 12 and a periphery thereof, the light transmitting resin 15 prevents transmission of water or exposure to outside air to prevent deterioration of the elements (the two light emitting elements 12 and the light receiving element 13), and allows transmission of light emitted from the two light emitting elements 12. A portion where the two light emitting elements 12 and the light receiving element 13 are provided is a light emitting/receiving portion.

The optoreflector 10 is built into an optosensor, and an example thereof is disclosed in Patent Document 1.

### PRIOR ART DOCUMENTS

### Patent Documents

[Patent Document 1] JP-B-4903980

### SUMMARY OF THE INVENTION

### Technical Problem

Incidentally, a mobile or wearable optosensor may also be used when light is incident on a skin for a long period of time, for example, for measuring a heart rate during long-distance running. Therefore, as the light emitting element 12, a high-power element such as a semiconductor laser cannot be used and an LED (light emitting diode) having an emission spectrum in a visible range or a near infrared range is generally used. However, light emitted from an LED has low coherence, is likely to be scattered, and also has wide directivity. Therefore, it is difficult to detect only reflected light from a blood vessel, and an effect of a DC signal generated by reflected light from a skin surface, a bone, or the like is unavoidable. Thus, a really required signal such as reflected light from a blood vessel is embedded in a DC signal, and there is a problem in that the detection accuracy deteriorates.

Further, in a mobile or wearable optosensor, a protective member (cover) that is disposed at a distance from a light emitting/receiving surface of the optoreflector 10 is present as compared to a stationary optosensor that is not exposed to rain or perspiration. Therefore, a part of light emitted from the light emitting element 12 in the optoreflector 10 is reflected from the protective member, and thus a DC signal is added. That is, not only a DC signal that is generated by reflected light from a skin surface, a bone, or the like but also a DC signal that is generated by reflected light from the protective member disposed near the optoreflector 10 is added.

FIG. 7 is a view schematically illustrating a state where a part of light emitted from the light emitting elements 12 in the optoreflector 10 is reflected from a cover 20 as the protective member. As illustrated in FIG. 7, a light emitting/receiving surface 10a of the optoreflector 10 faces the cover 20. A skin 30 of a biological tissue adheres to the cover 20. A part of light emitted from the light emitting elements 12 is reflected from an inner surface of the cover 20 or from an interface between the cover 20 and the skin 30 of the biological tissue without reaching a blood vessel 31 of the biological tissue, and is incident on the light receiving element 13. Reflected light L1 illustrated in FIG. 7 is light reflected from the inner surface of the cover 20, and reflected light L2 is light reflected from the interface between the cover 20 and the skin 30 of the biological tissue. This way, an unnecessary DC signal generated from a region other than the blood vessel 31 is incident on the light receiving element 13 and is detected by the light receiving element 13. Therefore, a really required signal, that is, a signal generated by reflected light from the blood vessel 31 is embedded in a DC signal, and the detection accuracy deteriorates.

FIG. 8 is a graph illustrating an output of the optoreflector 10 when a pulse is detected by the optosensor including the optoreflector 10. A pulse generated by light is detected by monitoring the amount of a change in hemoglobin in an artery. At this time, assuming that a maximum value of a sensor output is 100%, a signal generated by reflected light from the cover 20 and a signal generated by reflected light from the skin 30 of the biological tissue and the like account for most of the sensor output, and a signal that can be detected as a pulse is at an extremely low level of 0.2% or lower. Here, an intensity of a signal generated by reflected light from the blood vessel 31 periodically changes, and thus the signal will be referred to as an AC signal. On the other hand, a signal generated by reflected light from the cover 20 or reflected light from the skin 30 of the biological tissue and the like does not periodically change and is constant, and thus can be referred to as a DC signal. However, this DC signal is noise to the AC signal, and thus will be referred to as “DC noise”.

In a case where a pulse of a person is monitored while the person wearing the optosensor is running, an AC signal is further reduced by a body movement, which causes deterioration in detection accuracy. Among the signals illustrated in FIG. 8, a DC noise generated by reflected light from the cover 20 changes depending on the material or thickness of the cover 20. On the other hand, a DC noise generated by reflected light from a skin surface, a tissue, or the like is substantially constant. Accordingly, the most important issue for detecting a pulse with high accuracy is to reduce a DC noise mainly generated by reflected light from the cover 20 and to thereby improve an AC/DC ratio.

The present invention has been made in consideration of the above-described circumstances, and an object thereof is to provide an optosensor capable of preventing the generation of a DC noise by reflected light that hinders high-accuracy detection of a pulse.

### Solution to Problem

According to the invention, there is provided an optosensor that includes an optoreflector having a light emitting element and a light receiving element which are mounted on a substrate, a light transmitting resin sealing the light emitting element and the light receiving element, and a light blocking resin, in which a light blocking wall and an cave are formed between the light emitting element and the light receiving element with the light blocking resin, the cave being linked to the light blocking wall to narrow an upper light emitting surface of the light emitting element.

In addition, in the optosensor according to the invention, the cave narrows not only the upper light emitting surface of the light emitting element but also an upper light receiving surface of the light receiving element.

In addition, in the optosensor according to the invention, a light transmitting cover is disposed over a light emitting/receiving surface of the optoreflector.

In addition, in the optosensor according to the invention, the cave narrows not only the upper light emitting surface of the light emitting element but also an upper light receiving surface of the light receiving element, and a light transmitting cover is disposed over a light emitting/receiving surface of the optoreflector.

### Advantageous Effects of the Invention

According to the present invention, a part of light, which is emitted from the light emitting element and is reflected from the cover disposed near the optoreflector, is blocked by the cave and is not likely to reach the light receiving element. Therefore, the DC noise generated by reflected light from the cover is reduced, and the AC/DC ratio can be improved.

### BRIEF DESCRIPTION OF THE DRAWINGS

FIGS. 1(a) and 1(b) are views illustrating an optoreflector included in an optosensor according to an embodiment of the present invention.

FIG. 2 is a view schematically illustrating a state where the optoreflector according to the embodiment is sealed into the optosensor and used as a vital sensor.

FIG. 3 is a graph illustrating an example of a comparison between an AC/DC ratio of the optosensor including the optoreflector according to the embodiment and an AC/DC ratio of an optosensor including an optoreflector of the related art.

FIG. 4 is a table illustrating an example of the comparison between the AC/DC ratio of the optosensor including the optoreflector according to the embodiment and the AC/DC ratio of the optosensor including the optoreflector of the related art.

FIG. 5 is a view illustrating a modification example of the optoreflector according to the embodiment.

FIGS. 6(a) and 6(b) are views illustrating an optoreflector of the related art used in a mobile or wearable optosensor.

FIG. 7 is a view illustrating a state where a part of light emitted from light emitting elements in the optoreflector of the related art is reflected from a cover.

FIG. 8 is a graph illustrating an output of the optoreflector when a pulse is detected by the optosensor including the optoreflector of the related art.

### DESCRIPTION OF EMBODIMENTS

Hereinafter, a preferred embodiment for practicing the present invention will be described in detail with reference to the drawings.

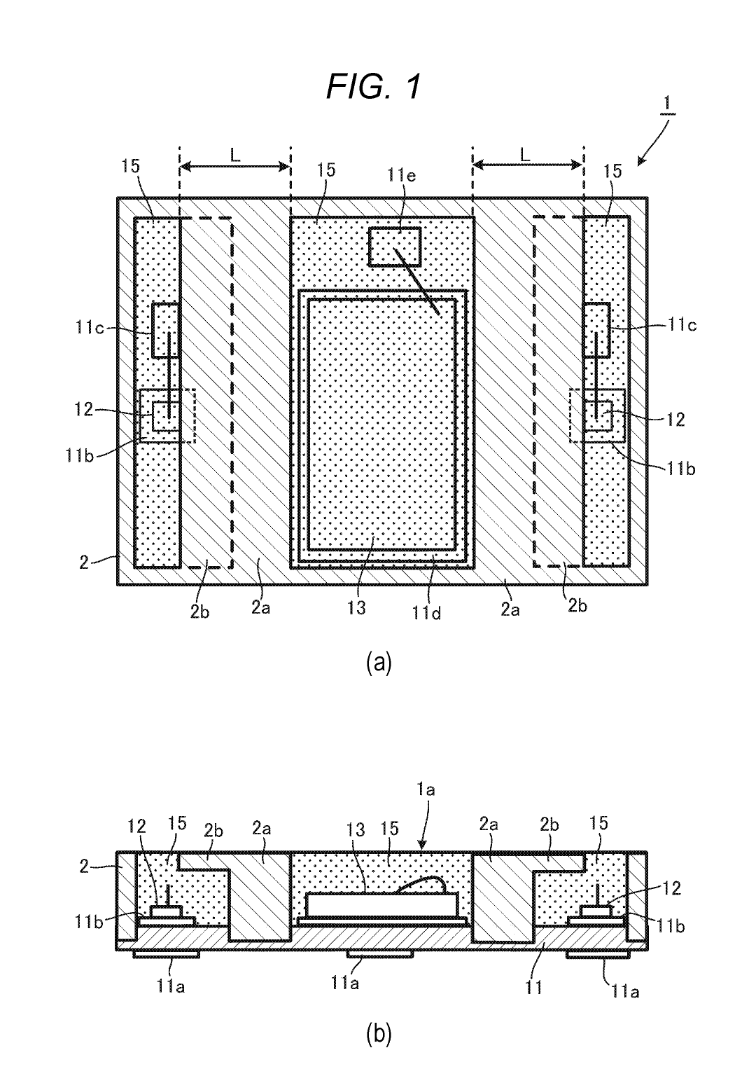

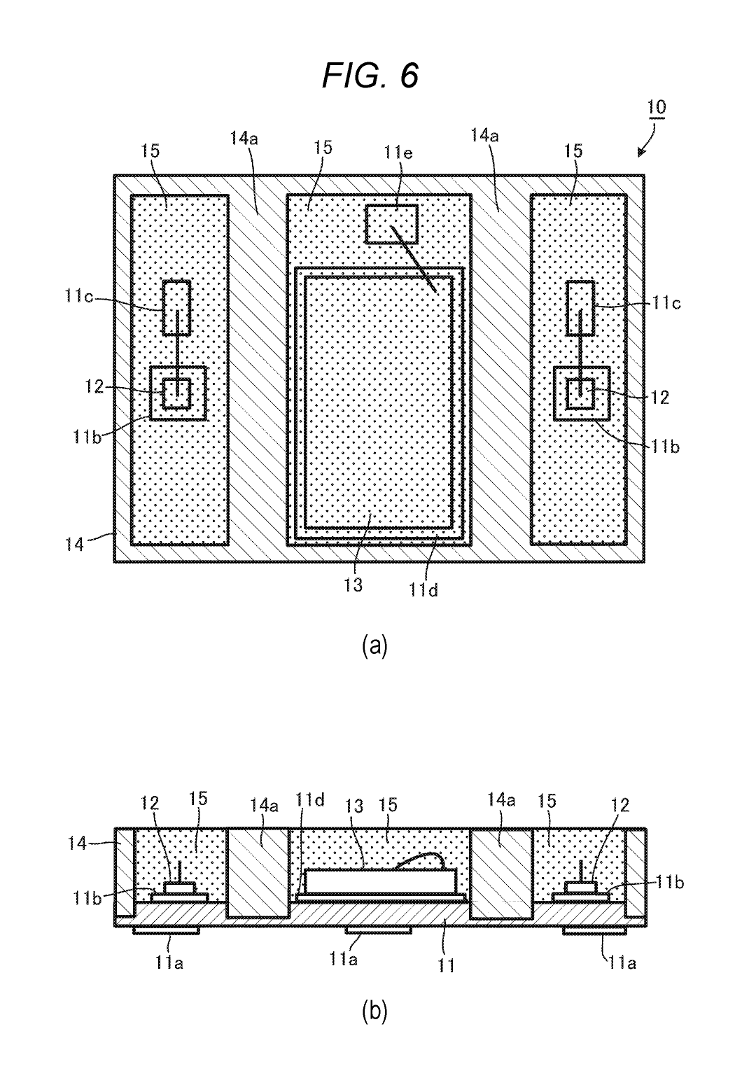

FIGS. 1(a) and 1(b) are views illustrating an optoreflector 1 included in an optosensor according to an embodiment of the invention. FIG. 1(a) is a plan view illustrating the optoreflector 1 when seen from a light emitting/receiving surface, and FIG. 1(b) is a cross-sectional view illustrating the optoreflector 1. In FIGS. 1(a) and 1(b), the same components as those in FIGS. 6(a) and 6(b) will be represented by the same reference numerals.

The optoreflector 1 according to the embodiment has a light blocking resin 2 having a shape which is partially different from that of the light blocking resin 14 of the optoreflector 10 of the related art. That is, an eave 2b is formed by the light blocking resin 2 to be linked to a light blocking wall 2a between one light emitting element 12 and the light receiving element 13 and to narrow an upper light emitting surface of the light emitting element 12, and another eave 2b is formed by the light blocking resin to be linked to another light blocking wall 2a between the other light emitting element 12 and the light receiving element 13 and to narrow an upper light emitting surface of the light emitting element 12. The light blocking wall 2a including the cave 2b is formed in a vertically and horizontally inverted L-shape as can be seen from the cross-sectional view of FIG. 1(b). The invention has a major characteristic in that the eave 2b is provided.

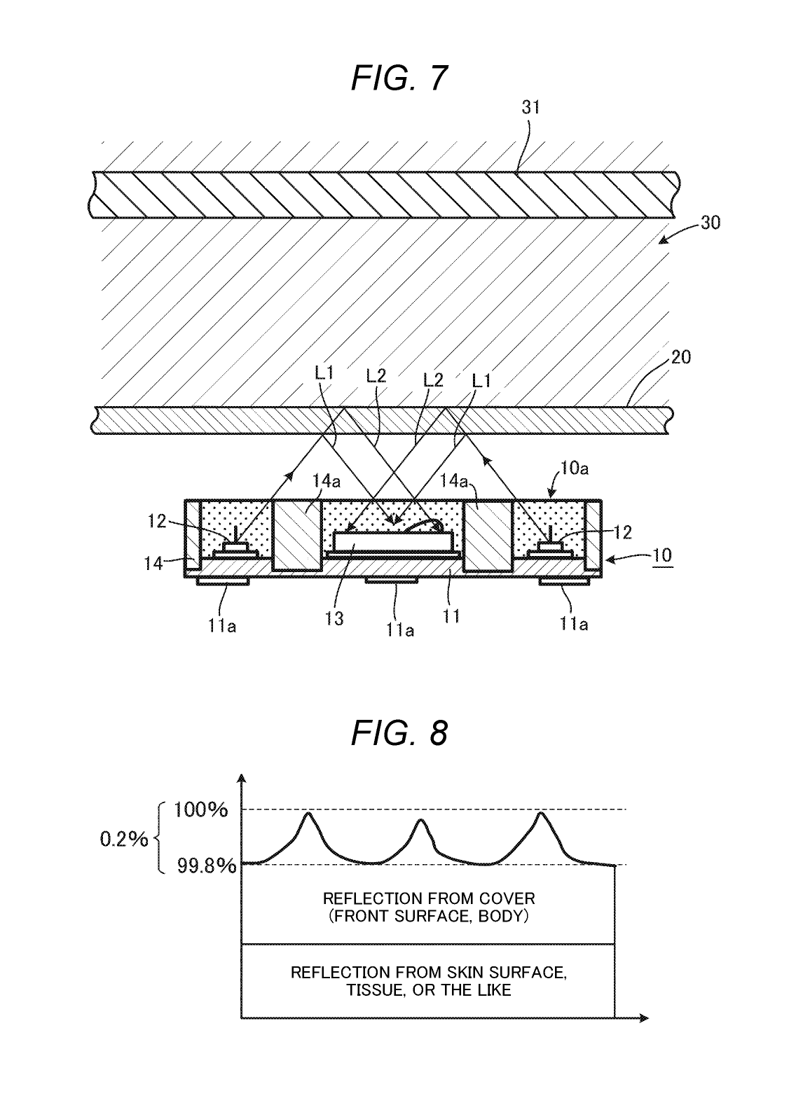

In a case where the optoreflector 1 according to the embodiment is configured as a mobile or wearable photosensor, the cover 20 illustrated in FIG. 7 is disposed near a light emitting/receiving surface 1a. The eave 2b of the light blocking resin 2 can block reflected light from the cover 20 or reflected light from the skin 30 (refer to FIG. 7) of the biological tissue from being incident on the light receiving element 13. In consideration of the distance between the light emitting element 12 and the light receiving element 13, the thickness of the light transmitting resin 15, and the position of the cover 20, the protrusion amount of the eave 2b (that is, the length extending toward the light emitting element 12) is adjusted such that the amount of light reflected from the blood vessel 31 (refer to FIG. 7) of the biological tissue is the maximum.

In addition, in FIG. 1(a), a total width L of the light blocking wall 2a and the eave 2b in a plan view is appropriately determined based on the size of the light receiving element 13. In consideration of an angle of an optical axis connecting a center of the light emitting element 12 and an end portion of the eave 2b, the width L is adjusted such that light emitted from the light emitting element 12 does not interfere with incidence of reflected light from the blood vessel 31 as much as possible and does not interfere with incidence of reflected light from the cover 20 or the skin 30 of the biological tissue as much as possible.

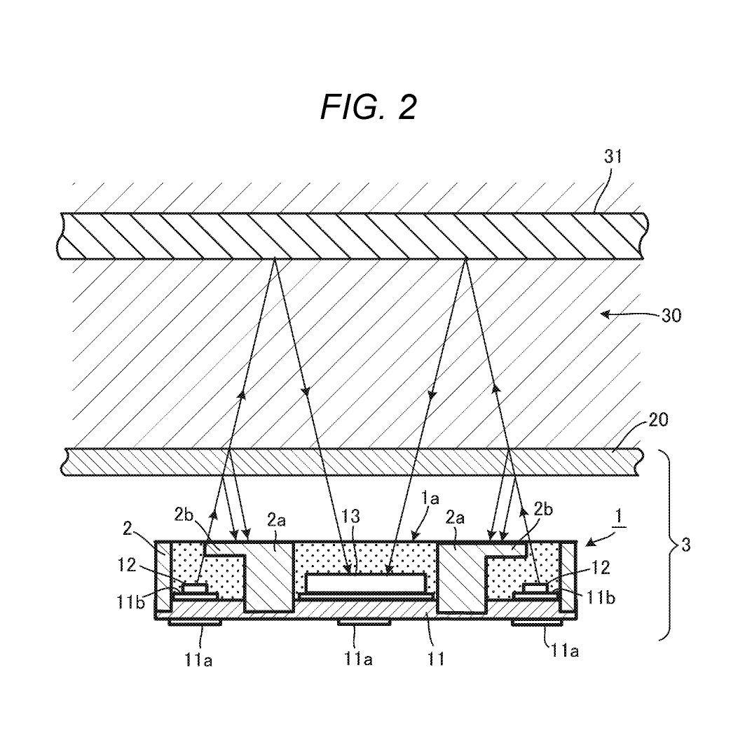

FIG. 2 is a view schematically illustrating a state where the optoreflector 1 according to the embodiment is sealed into an optosensor 3 and used as a vital sensor. As illustrated in FIG. 2, the cave 2b blocks a part of light from being incident on the light receiving element 13, the part of light being emitted from the light emitting elements 12 and being reflected from the inner surface of the cover 20 or from the interface between the cover 20 and the skin 30 of the biological tissue without reaching the blood vessel 31. In the optoreflector 1 according to the embodiment, the distance between the light emitting element 12 and the light receiving element 13 is wider than that of the optoreflector 10 of the related art. By widening the distance between the light emitting element 12 and the light receiving element 13, the amount of reflected light from the cover 20 or the amount of reflected light from the skin 30 of the biological tissue can be reduced, and the AC/DC ratio can be increased. However, in a case where the distance between the light emitting element 12 and the light receiving element 13 is excessively wide, the shape of the optosensor 3 is excessively large. Therefore, the shape of the optosensor 3 may be determined according to the use.

By appropriately adjusting the protrusion amount of the cave 2b, the distance between the light emitting element 12 and the light receiving element 13, and the thickness of the light transmitting resin 15, respectively, the angle of the optical axis connecting an end portion of the cave 2b and the center of the light emitting element 12 can be adjusted, and directivity can be imparted to light such that the light is reflected from the blood vessel 31 in a body and is incident on the light receiving element 13.

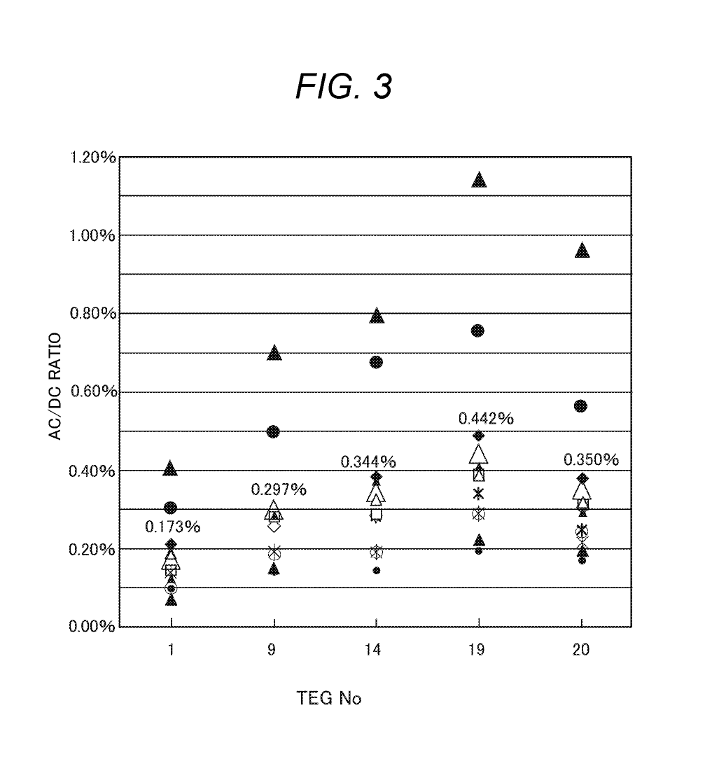

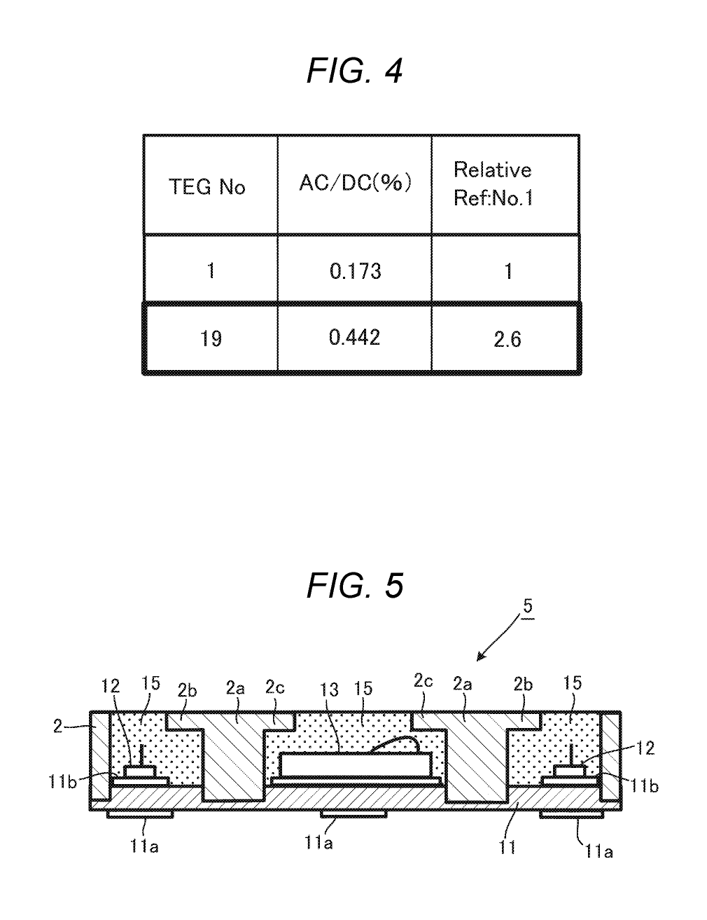

FIGS. 3 and 4 are a graph and a table illustrating an example of a comparison between an AC/DC ratio of the optosensor including the optoreflector 1 according to the embodiment and an AC/DC ratio of an optosensor including an optoreflector 10 of the related art. In FIG. 3, the horizontal axis represents TEG NO. (Test Element Group No.; so-called, sample No.). The vertical axis represents an AC/DC ratio. Among TEG No. 1, 9, 14, 19, and 20, No. 1 represents the AC/DC ratio of the optoreflector 10 of the related art, and No. 19 represents the AC/DC ratio of the optoreflector 1 according to the embodiment. FIG. 3 illustrates the results of comparing TEG No. 1 and the TEG No. 19 to each other regarding each of 12 subjects. An average value (indicated by “Δ”) of the AC/DC ratio of the optoreflector 10 of the related art of TEG No. 1 is “0.173%”, and an average value of the AC/DC ratio of the optoreflector 1 according to the embodiment of TEG No. 19 is “0.442%”. The reason why the AC/DC ratios of the 12 subjects are different from each other is that the thickness of an arm or the thickness of a blood vessel varies depending on people.

FIG. 4 illustrates that the AC/DC ratio of the optosensor including the optoreflector 10 of the related art of TEG No. 1 is “0.173%”, that the AC/DC ratio of the optosensor including the optoreflector 1 of the related art of TEG No. 19 is “0.442%”, and that in a case where the AC/DC ratio “0.173%” of the optosensor including the optoreflector 10 of the related art of TEG No. 1 is set as a reference “1”, a relative value of the optosensor including the optoreflector 1 according to the embodiment is “2.6”. This way, it can be seen that the AC/DC ratio of the optosensor including the optoreflector 1 according to the embodiment is improved by 2.6 times as compared to the AC/DC ratio of the optosensor including the optoreflector 10 of the related art.

This way, the optoreflector 1 according to the embodiment adopts a structure in which the light blocking wall 2a and the L-shaped cave 2b are formed, the light blocking wall being formed to appropriately adjust the distance between the light receiving element 13 and the light emitting element 12 and to block the light receiving element and the light emitting element, the L-shaped eave being linked to the light blocking wall 2a to narrow a part of the light transmitting resin 15 on the light emitting element 12. Therefore, a DC noise that cannot be blocked by the optoreflector 10 having the structure of the related art illustrated in FIGS. 6(a) and 6(b) can be effectively reduced, the DC noise being generated by reflected light from the cover 20 or reflected light from the skin 30 of the biological tissue. As a result, the detection accuracy of a pulse can be improved. By providing the cave 2b, the DC noise generated by reflected light from the cover 20 or reflected light from the skin 30 of the biological tissue can be reduced, and the following effect can also be obtained using the cave 2b. The amount of light guided to the blood vessel 31 present in a deep portion of a body can be made to be more than the amount of light reflected from other portions, and the level of the AC signal generated by reflected light from the blood vessel 31 can be increased.

In the light blocking resin 2 of the optoreflector 1 according to the embodiment, the shape of the light blocking wall 2a including the cave 2b in a cross-sectional view is a L-shape but may also be a T-shape. That is, in addition to the cave 2b that extends toward the light emitting element 12, an cave that extends toward the light receiving element 13 may be further formed. FIG. 5 is a cross-sectional view illustrating an optoreflector 5 according to a modification example of the optoreflector 1. FIG. 5 is a cross-sectional view corresponding to FIG. 1(b). As illustrated in, FIG. 5, in the light blocking resin 2 of the optoreflector 5 according to the modification example, a T-shaped structure is adopted by forming an cave 2c that extends toward the light receiving element 13. With the optoreflector 5, the degree of freedom in design can be improved.

The number of light emitting elements 12 is not limited to two and may be three or more.

Hereinabove, the embodiment has been described. However, various modifications can be made within the scope of the invention. For example, the embodiment, the measurement position is a wrist of a human body. However, the measurement position may be an external acoustic opening of a human body. In addition, the invention is also applicable to an animal instead of a human body.

### INDUSTRIAL APPLICABILITY

The present invention has an effect of providing an optosensor capable of preventing the generation of a DC noise by reflected light that hinders high-accuracy detection of a pulse, and can be used for measuring biological information such as a pulse wave, a heart rate, a blood oxygenation level, or the like in the healthcare industry.

### REFERENCE SIGNS LIST

1, 5: Photoreflector

1a: Light emitting/receiving surface

2: Light blocking resin

2a : Light blocking wall

2b, 2c: Pave

3: Photosensor

11: Substrate

11a: External electrode

11b, 11d: Die pad

11c, 11e: Bonding pad

12: Light emitting element

13: Light receiving element

15: Light transmitting resin

20: Cover

30: Skin of biological tissue

31: Blood vessel

1. An optosensor comprising:
an optoreflector having a light emitting element and a light receiving element which are mounted on a substrate, a light transmitting resin sealing the light emitting element and the light receiving element, and a light blocking resin,
wherein a light blocking wall and an cave are formed between the light emitting element and the light receiving element with the light blocking resin, the cave being linked to the light blocking wall so as to narrow an upper light emitting surface of the light emitting element.

2. The optosensor according to claim 1,
wherein the cave narrows an upper light receiving surface of the light receiving element, in addition to the upper light emitting surface of the light emitting element.

3. The optosensor according to claim 1,
wherein a light transmitting cover is disposed over a light emitting surface and a light receiving surface of the optoreflector.

4. The optosensor according to claim 1,
wherein the cave narrows an upper light receiving surface of the light receiving element, in addition to the upper light emitting surface of the light emitting element, and a light transmitting cover is disposed over a light emitting surface and a light receiving surface of the optoreflector.
